(19) United States
(12) Patent Application Publication
(10) Pub. No.: US 2006/0154300 A1
(43) Pub. Date: Jul. 13, 2006

(54) Biomolecule bonding detection apparatus using wireless antenna and method thereof

(76) Inventors: Tae-sik Park, Suwon-si (KR); Young-il Kim, Suwon-si (KR); Jung-ho Kang, Suwon-si (KR)

Correspondence Address: CANTOR COLBURN, LLP, 55 GRIFFIN ROAD SOUTH, BLOOMFIELD, CT, 06002, US

(21) Appl. No.: 11/327,032

(22) Filed: Jan. 5, 2006

(30) Foreign Application Priority Data
Jan. 7, 2005 (KR) 10-2005-0001850

(51) Int. Cl. C12M 1/34; G01N 33/53; G08C 19/04

(52) U.S. Cl. 435/7.1; 435/287.2; 340/870.11

(57) ABSTRACT

Disclosed is a biomolecule binding detection apparatus and a method thereof, which detects biomolecule bonding by measuring change in characteristics of a test signal transmitted by an antenna on which probe biomolecules are immobilized. The biomolecule bonding detection apparatus includes a transmitting unit on which probe biomolecules are immobilized, the probe biomolecules configured for detection of specific gene information included in sample biomolecules to be analyzed, the transmitting unit further configured for signal transmission. A receiving unit is configured for detecting biomolecule bonding by comparison of selected characteristics of a test signal transmitted from the transmitting unit before and after mixing of the probe biomolecules and the sample biomolecules.

This application claims priority under 35 U.S.C. § 119 to Korean Patent Application No. 2005-1850, filed on Jan. 7, 2005, the entire content of which is incorporated herein by reference.

### BACKGROUND OF THE INVENTION

1. Field of the Invention

The present invention relates to a biomolecule bonding detection apparatus using a wireless antenna and a method thereof, and more particularly a biomolecule bonding detection apparatus using a wireless antenna and a method thereof, which detects biomolecule bonding by measuring a change in signals transmitted through a wireless antenna on which probe biomolecules are immobilized.

2. Description of the Related Art

A biomolecule chip is a biological microchip having biomolecules immobilized on a substrate. Biomolecule chips can be categorized based on the type of immobilized biomolecule, for example, DNA chips, protein chips, etc. A biomolecule which is immobilized on a chip and binds with a target biomolecule in a sample is called a probe. Biomolecule chip-related technical fields in development include, for example: biomolecule immobilization techniques for immobilizing biomolecules on a substrate, techniques for bonding immobilized biomolecules on a biomolecule chip with components of a sample, and biomolecule detection techniques for detecting the existence and the kind of biomolecules based on the analysis of a biomolecule chip where unknown biomolecules are immobilized.

Presently, there are several signal detection techniques in existence for the detection of biomolecule bonding. Broadly stated, these techniques may be classified into categories such as, for example, optical biomolecule bonding detection, chemical biomolecule bonding detection, electrical biomolecule bonding detection, mechanical biomolecule detection, etc.

A conventional optical biomolecule bonding detection method is generally implemented by labeling a biomolecule sample with a fluorescent material, and then mixing the biomolecule sample with the probe biomolecule on the biomolecule chip such that ligands in the sample can bind with the probe biomolecule. The results are then analyzed using a fluorescent detection device so as to optically determine the amount of bonding with the probe biomaterials. However, this method may cause loss or contamination of the sample biomolecules due to the requirement for a preprocess for bonding the sample biomolecules with the fluorescent materials prior to the bonding reaction between the probe biomolecules and the sample biomolecules. In addition, an expensive, complex optical reader and measurement device are needed to detect and analyze the bonding reaction between the probe biomolecules and the sample biomolecules. Moreover, the optical detection devices are not easily miniaturized, and do not provide digitized outputs.

Conventional mechanical biomolecule bonding detection methods utilize devices such as, for example, a cantilever, a surface acoustic wave (SAW) biosensor, a SPM (Scanning Probe Microscope) or the like. Where a cantilever is used, intermolecular coherences before and after bonding between the probe biomolecules and sample biomolecules are measured to detect the presence or absence of biomolecule bonding. However, in order to measure the intermolecular cohesion, the deflection of the cantilever must be accurately determined, which requires auxiliary equipment such as a laser device.

Detection through a SAW biosensor utilizes the input of a signal at a specific frequency. The presence and absence of biomolecule bonding is then detected by observing filtering variation of the SAW filter caused by reaction between sample biomolecules and probe biomolecules immobilized on the SAW filter. In addition, the biomolecule bonding detection method using an SPM is disadvantageous in that it requires auxiliary equipment such as a laser device and a photodiode.

Conventional chemical biomolecule bonding detection methods detect the presence and absence of biomolecule bonding by analyzing the degree of electrochemical reaction of other chemical materials on an electrode on which probe biomolecules and sample biomolecules bind with each other. However, this method provides an inferior detection capability in comparison to optical biomolecule bonding detection methods.

In addition, conventional electrical biomolecule bonding detection methods may use structures such as, for example, a trench-type capacitance element or a planar-type capacitance element. However, since capacitance is proportional to cross sectional area and inversely proportional to thickness, it is difficult to design and form a capacitance element having an increased cross sectional area together while also ensuring efficient bio processing.

In particular, a detection method using a trench-type capacitor provides a method of reducing thickness and increasing cross sectional area of the capacitor by forming a deep trench, however it is not useful for bio processing since the resulting gap is very small. In contrast, a biomolecule bonding detection method using a capacitor in which a capacitance element is formed with a comb shape on a plane is disadvantageous in that only a relatively small number of capacitance elements are formed, since the metal film has a small thickness, and the detection sensitivity for biomolecules bonding is poor.

### SUMMARY OF THE INVENTION

Therefore, it is an object of the present invention to provide a biomolecule bonding detection apparatus and a method thereof, which detects biomolecule bonding by utilizing the change in characteristics of signal transmitted by an antenna having probe biomolecules bonded thereon, depending on the presence and/or absence of biomolecule bonding of sample biomolecules to the probe biomolecules.

In order to accomplish the above and other objects, the present invention provides a biomolecule bonding detection apparatus, which includes a transmitting unit on which probe biomolecules are immobilized, the probe biomolecules configured for detection of specific gene information included in sample biomolecules to be analyzed, the transmitting unit further configured for signal transmission; and a receiving unit configured for detecting biomolecule bonding by comparison of selected characteristics of a test signal transmitted from the transmitting unit before and after mixing of the probe biomolecules and the sample biomolecules.

Preferably, the transmitting unit includes a first antenna configured for transmitting the test signal, the probe biomolecules being immobilized thereon; and a generation unit connected to the first antenna, the generation unit configured to generate the signal transmitted by the first antenna.

Also preferably, the receiving unit includes a second antenna configured for receiving the test signal transmitted by the transmitting unit; and an analyzing unit connected to the second antenna, the analyzing unit configured to analyze selected characteristics of the test signal received by the second antenna, wherein the analyzing unit detects biomolecule bonding by comparing the selected characteristics of the test signal before and after mixing of the probe biomolecules and the sample biomolecules.

In an embodiment, the selected characteristics of the signal include one or more of frequency, amplitude, energy and phase.

In one embodiment, the first and second antennas are RF antennas.

In another embodiment, a biomolecule binding detection method includes immobilizing probe biomolecules on a transmitting unit for detection of specific gene information included in sample biomolecules to be analyzed; analyzing selected characteristics of a test signal transmitted from the transmitting antenna and received by a receiving unit; mixing the probe biomolecules with the sample biomolecules; and analyzing characteristics of the received signal after the biomolecule mixing, and detecting biomolecule bonding by detecting a change of the selected characteristics of the analyzed signal after the biomolecule mixing.

In one embodiment, the characteristics of the test signal include one or more of frequency, amplitude, energy and phase.

In another embodiment, the method further includes immobilizing the probe biomolecules on a first antenna included in the transmitting unit, the first antenna configured to transmit the test signal, the test signal generated by a generation unit connected thereto; and receiving the test signal by a second antenna included in the receiving unit, and analyzing the received test signal with an analyzing unit included within the receiving unit.

In another embodiment, the first antenna and the second antenna are RF antennas.

### BRIEF DESCRIPTION OF THE DRAWINGS

The above aspects and features of the present invention will be more apparent by describing certain embodiments of the present invention with reference to the accompanying drawings, in which:

FIG. 1 is a perspective view illustrating a biomolecule binding detection apparatus using a wireless antenna according to an embodiment of the present invention;

FIGS. 2A to 2C are perspective views illustrating a biomolecule binding detection method using the biomolecule binding detection apparatus of FIG. 1; and

FIG. 3 is a flowchart for illustrating a biomolecule binding detection method using a wireless antenna according to an embodiment of the present invention.

### DETAILED DESCRIPTION OF THE INVENTION

The invention is described more fully hereinafter with reference to the accompanying drawings, in which exemplary embodiments of the invention are shown. This invention may, however, be embodied in many different forms and should not be construed as limited to the exemplary embodiments set forth herein. Rather, these embodiments are provided so that this disclosure will be thorough and complete, and will fully convey the scope of the invention to those skilled in the art. In the drawings, the size and relative sizes of layers and regions may be exaggerated for clarity.

It will be understood that when an element or layer is referred to as being “on” or “connected to” another element or layer, the element or layer can be directly on or connected to another element or layer or intervening elements or layers. In contrast, when an element is referred to as being “directly on” or “directly connected to” another element or layer, there are no intervening elements or layers present. Like numbers refer to like elements throughout. As used herein, the term “and/or” includes any and all combinations of one or more of the associated listed items.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Embodiments of the invention are described herein with reference to schematic illustrations of idealized embodiments (and intermediate structures) of the invention. As such, variations from the shapes of the illustrations as a result, for example, of manufacturing techniques and/or tolerances, are to be expected. Thus, embodiments of the invention should not be construed as limited to the particular shapes of regions illustrated herein but are to include deviations in shapes that result, for example, from manufacturing. Also, well-known functions or constructions are not described in detail since they would obscure the invention in unnecessary detail.

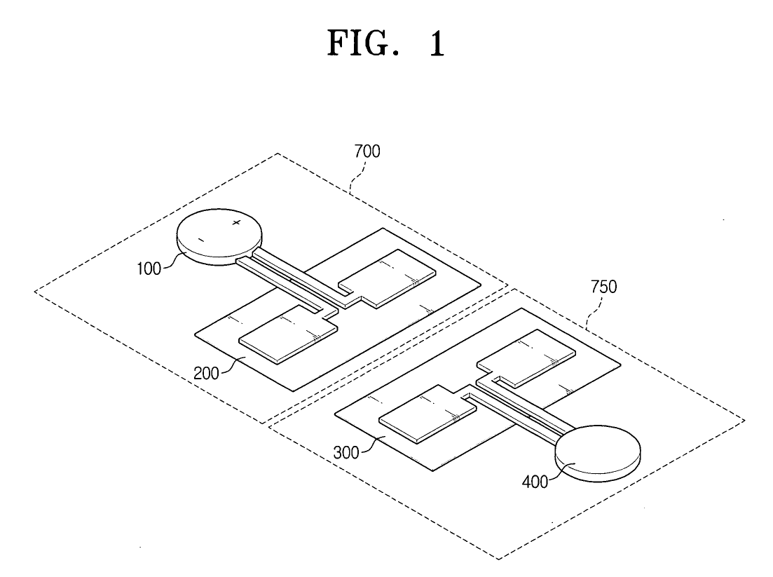

FIG. 1 illustrates a biomolecule binding detection apparatus using a wireless antenna according to an embodiment of the present invention.

As shown in FIG. 1, the biomolecule binding detection apparatus includes a transmitting unit 700 configured for transmitting a signal, the transmitting unit 700 having probe biomolecules immobilized thereon, and a receiving unit 750 configured for receiving a signal from the transmitting unit 700 and analyzing characteristics of the received signal. More specifically, the transmitting unit 700 includes a generation unit 100 and a first antenna 200 upon which probe biomolecules are immobilized, while the receiving unit 750 includes a second antenna 300 and an analyzing unit 400.

In operation, the generation unit 100 generates a signal to be transmitted through an antenna. The generated signal is transmitted to the first antenna 200.

The first antenna 200 is connected to the generation unit 100 and transmits the signal generated by the generation unit 100 to the second antenna 300. The probe biomolecules immobilized on the first antenna 200 allow detection of specific gene information included in a sample biomolecule to be analyzed.

The second antenna 300 may transmit and receive a signal to/from the first antenna 200, and transmits a signal transmitted from the first antenna 200 to the analyzing unit 400.

The analyzing unit 400 is connected to the second antenna 300, and analyzes characteristics of the signal received from the first antenna 200. The specific characteristic(s) of the signal to be analyzed may include one or more of signal frequency, amplitude, phase, and energy. In addition, the analyzing unit 400 analyzes the characteristics of the signal received by the second antenna 300 both before and after the probe biomolecules are bound with the sample biomolecules. If the signal characteristics before and after binding are different from each other, the analyzing unit 400 determines that biomolecule bonding has occurred.

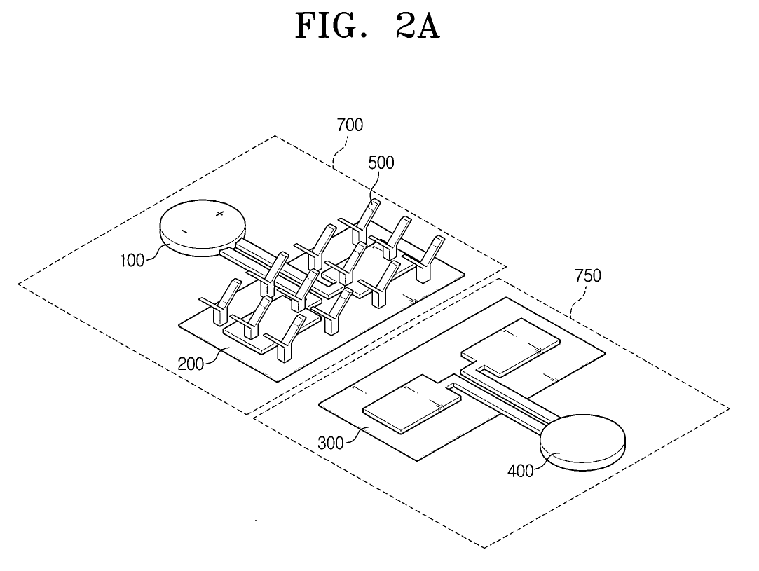

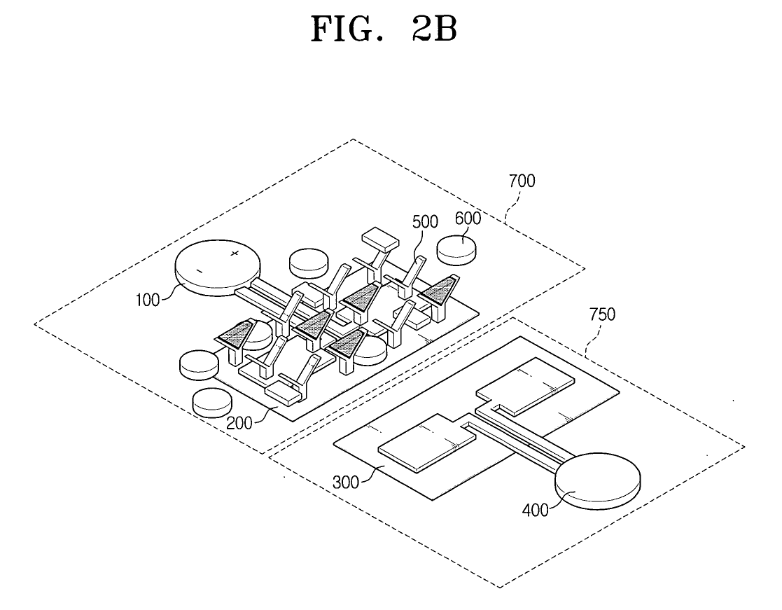

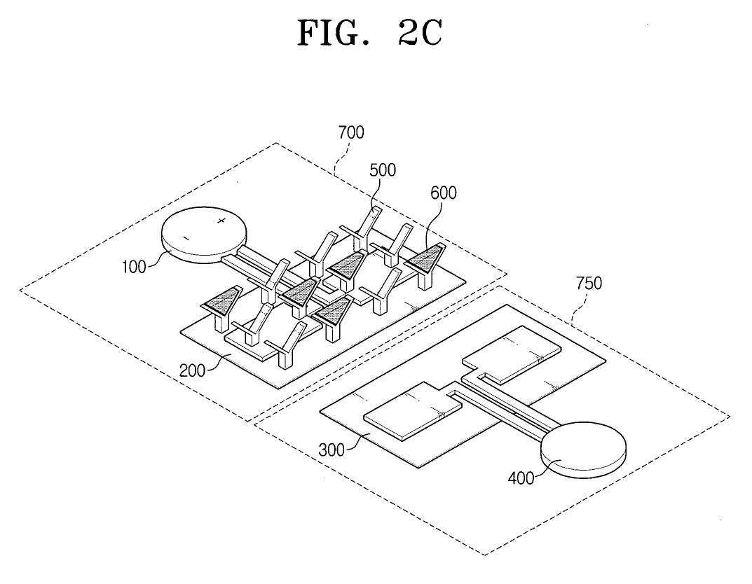

FIGS. 2A to 2C are diagrams illustrating a biomolecule binding detection method using the biomolecules binding detection apparatus of FIG. 1.

FIG. 2A schematically depicts probe biomolecules 500 that are immobilized on the first antenna 200, while FIG. 2B schematically depicts the probe biomolecules 500 immobilized on the first antenna 200 bound with sample biomolecules 600. In addition, FIG. 2C schematically depicts the cleaning (removal) of those sample biomolecules 600 that are not bound with the probe biomolecules 500.

Referring to FIG. 2A, the probe biomolecules 500 are immobilized on the first antenna 200, and characteristics such as frequency, amplitude and phase of a signal transmitted to the second antenna 300 from the first antenna 200 are analyzed. The signal is first analyzed prior to any binding of the probe biomolecules 500 with sample biomolecules

Referring to FIG. 2B, certain of the sample biomolecules 600 to be analyzed are mixed with the probe biomolecules 500 immobilized on the first antenna 200. Any of sample biomolecules 600 that are not subsequently bound with the probe biomolecules 500 are then removed.

Referring to FIG. 2C, after the biomolecule binding, the characteristics of the signal transmitted from the first antenna 200 to the second antenna 300 are once again analyzed. In the event that probe biomolecules 500 are in fact bound with the sample biomolecules 600, the characteristics of the signal transmitted to the second antenna 300 are changed as a result. Thus, if the characteristics of the signal transmitted to the second antenna 300 before the biomolecule mixing are changed with respect to the characteristics transmitted after the biomolecule mixing, the analyzing unit 400 then determines that the probe biomolecules 500 immobilized on the first antenna 200 are bound with the sample biomolecules 600.

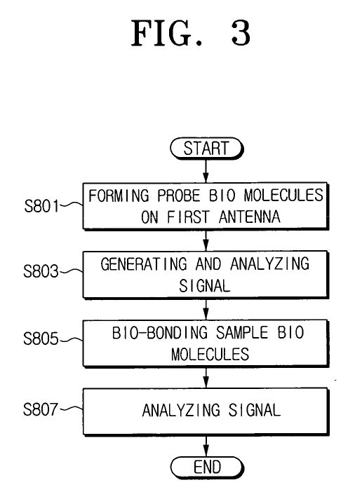

FIG. 3 is a flowchart illustrating a biomolecule bonding detection method using a wireless antenna according to an embodiment of the present invention.

Referring to FIG. 3, the method depicted therein utilizes a biomolecule binding detection apparatus including a transmitting unit 700 having a first antenna 200 on which probe biomolecules are immobilized, and a receiving unit 750 for analyzing characteristics of a signal received from the transmitting unit 700.

First, probe biomolecules 500 are immobilized on the first antenna 200 of the transmitting unit 700 so as to be configured to detect specific gene information included in a sample biomolecule to be analyzed (S801).

Subsequently, a signal to be transmitted to the receiving unit 750 through the first antenna 200 is generated, and the signal transmitted to the receiving unit 750 is analyzed (S803). The signal generated by the generation unit 100 is transmitted to an antenna of the receiving unit 750 through the first antenna 200, and characteristics such as (for example) frequency, amplitude, phase and energy of the transmitted signal are analyzed. The information related to the characteristics of the analyzed signal is stored in the analyzing unit 400 of the receiving unit 750.

Subsequently, the probe biomolecules 500 immobilized on the first antenna 200 are mixed with the sample biomolecules 600 (S805). Among the sample biomolecules 600, those having a ligand with sufficient complementarity to the probe biomolecules 500 are bound with the probe biomolecules 500. Sample biomolecules 600 not having a ligand with sufficient complementarity to the probe biomolecules 500 are removed.

Subsequently, after the probe biomolecules 500 and the sample biomolecules 600 are mixed, the characteristics of the signal transmitted to the receiving unit 750 through the first antenna 200 are again analyzed (S807) in the same manner prior to mixing. If the characteristics of the signal analyzed before biomolecule mixing are different from those after mixing, it is determined that biomolecule bonding has occurred on the first antenna 200. As the probe biomolecules 500 are bound with the sample biomolecules 600, the characteristics of the signal transmitted through the first antenna 200 are changed due to the bonding on the first antenna 200.

As described above, the present invention immobilizes probe biomolecules on a wireless antenna, thus the presence and absence of biomolecule binding by measuring change of signal characteristics transmitted through a wireless antenna before and after biomolecules binding may be easily detected.

Accordingly, the apparatus for detecting biomolecule binding may be easily manufactured through a simple structure.

The foregoing embodiments and advantages are merely exemplary and are not to be construed as limiting the present invention. The present teaching can be readily applied to other types of apparatuses. Also, the description of the embodiments of the present invention is intended to be illustrative, and not to limit the scope of the claims, and many alternatives, modifications, and variations will be apparent to those skilled in the art.

What is claimed is:

1. A biomolecule bonding detection apparatus, comprising:
a transmitting unit on which probe biomolecules are immobilized, the probe biomolecules configured for detection of specific gene information included in sample biomolecules to be analyzed, the transmitting unit further configured for signal transmission; and
a receiving unit configured for detecting biomolecule bonding by comparison of selected characteristics of a test signal transmitted from the transmitting unit before and after mixing of the probe biomolecules and the sample biomolecules.

2. The biomolecule bonding detection apparatus according to claim 1, wherein the transmitting unit includes:
a first antenna for configured for transmitting the test signal, the probe biomolecules being immobilized thereon; and
a generation unit connected to the first antenna, the generation unit configured to generate the signal transmitted by the first antenna.

3. The biomolecule bonding detection apparatus according to claim 2, wherein the receiving unit includes:
a second antenna configured for receiving the test signal transmitted by the transmitting unit; and
an analyzing unit connected to the second antenna, the analyzing unit configured to analyze characteristics of the test signal received by the second antenna, wherein the analyzing unit detects biomolecule bonding by comparing the selected characteristics of the test signal before and after mixing of the probe biomolecules and the sample biomolecules.

4. The biomolecule bonding detection apparatus according to claim 1, wherein the selected characteristics of the signal includes one or more of frequency, amplitude, energy and phase.

5. The biomolecule bonding detection apparatus according to claim 3, wherein the first and second antennas are RF antennas.

6. A biomolecule bonding detection method, comprising:
immobilizing probe biomolecules on a transmitting unit for detection of specific gene information included in sample biomolecules to be analyzed;
analyzing selected characteristics of a test signal transmitted from the transmitting unit and received by a receiving unit;
mixing the probe biomolecules with the sample biomolecules; and
analyzing the selected characteristics of the test signal after the biomolecule mixing, and detecting biomolecule bonding by detecting a change of the selected characteristics of the test signal after the biomolecule mixing.

7. The biomolecule bonding detection method according to claim 6, wherein the selected characteristics of the test signal includes one or more of frequency, amplitude, energy and phase.

8. The biomolecule bonding detection method according to claim 6, further comprising:
immobilizing the probe biomolecules on a first antenna included in the transmitting unit, the first antenna configured to transmit the test signal, the test signal generated by a generation unit connected thereto; and
receiving the test signal by a second antenna included in the receiving unit, and analyzing the received test signal with an analyzing unit included within the receiving unit.

9. The biomolecule bonding detection method according to claim 8, wherein the first antenna and the second antenna are RF antennas.

10. The biomolecule bonding detection method according to claim 6, further comprising removing unbonded sample biomolecules following the mixing.
